(19) United States
(12) Patent Application Publication
(10) Pub. No.: US 2025/0228582 A1
(43) Pub. Date: Jul. 17, 2025

(54) MEDICAL DEVICES AND RELATED SYSTEMS AND METHODS OF USE

(71) Applicant: Boston Scientific Medical Device Limited, Galway (IE)

(72) Inventors: Aditya DHANOTIYA, Indore, (IN); Yogesh Kishor VIKHARANKAR, Pune (IN); Sucheta MISHRA, Pimpri, (IN); Snehal PATIL, Pune, (IN); Hitendra PUROHIT, Vadodara, (IN)

(73) Assignee: Boston Scientific Medical Device Limited, Galway (IE)

(21) Appl. No.: 19/016,177

(22) Filed: Jan. 10, 2025

Related U.S. Application Data
(60) Provisional application No. 63/620,334, filed on Jan. 12, 2024.

(51) Int. Cl. A61B 17/221; A61B 1/012; A61B 1/307; A61B 17/00; A61B 17/22; A61B 90/00

(52) U.S. Cl. CPC A61B 17/221; A61B 1/012; A61B 1/307; A61B 17/00234; A61B 90/03; A61B 2017/00296; A61B 2017/0042; A61B 2017/00477; A61B 2017/22051; A61B 2017/22069; A61B 2090/034

(57) ABSTRACT

Medical systems are described, including systems comprising a first medical device, e.g., which may comprise a handle assembly including an actuator; a shaft extending longitudinally from the handle assembly to a distal end; and a wire assembly extending through the shaft. The wire assembly may comprise a proximal portion coupled to the handle assembly; a first distal extension extending distally from the proximal portion, and a second distal extension extending distally from the proximal portion. The shaft may include a proximal section extending around the proximal portion, a first distal section extending around the first extension, a second distal section extending around the second extension, and a basket portion. The basket portion may be configured to transition from a first retracted configuration to a first expanded configuration.

### CROSS-REFERENCE TO RELATED APPLICATIONS

This application claims the benefit of priority under 35 U.S.C. § 119 from U.S. Provisional Application No. 63/620,334, filed Jan. 12, 2024, which is incorporated by reference herein in its entirety.

### TECHNICAL FIELD

Various aspects of this disclosure relate generally to medical devices, systems, and procedures. Particular aspects relate to medical devices and systems for removing material, e.g., obstructions, from one or more body lumens and/or passageways, among other aspects.

### BACKGROUND

Kidney stones (known as ureteral calculi in medical terminology) are a significant burden on society and the health care system. Kidney stones form in the body when the amount of various minerals in urine exceeds an amount that can be eliminated (the metastable limit), and the excess minerals form a precipitate. Most kidney stones are comprised of calcium and oxalate, though uric acid, struvite, cysteine, and other stone compositions are also commonly found. Kidney stones may form in parts of the kidney, and may travel through parts of the urinary tract towards the bladder. In some cases, a kidney stone may become stuck en route to the bladder in the ureter, which can result in increased internal pressure in the kidney and may cause a pain known as renal colic.

Pain relief from kidney stones typically occurs instantly after stone passage or removal. Current kidney stone removal methods have at least some shortcomings. Ureteroscopy (URS) is a procedure in which a urologist inserts an endoscope up the urethra, into the bladder, and up the ureter to the site of the stone. Using a laser, the urologist fragments the stone into smaller pieces and retracts the fragments with a basket. The procedure requires general anesthesia, high skill level from the urologist, and anywhere from 20 minutes to one hour. The typical amount of manipulation of the ureteroscope within the ureter during the procedure, as well as the overall time spent in the ureter, can induce ureteral stricture (blockages of the ureter caused by a process similar to scarring). The procedure outcome is generally highly effective, but due to the risk of complications and required general anesthesia, URS is generally recommended only for stones that are 8-15 mm in size.

### SUMMARY

The present disclosure relates to, among other things, medical devices and systems for removing obstructions from body lumens and/or passageways, and related methods of use. Aspects of this disclosure may decrease the time to remove an obstruction, such as a kidney stone, from a patient's body. Further, for example, aspects of the present disclosure may reduce the level of skill of a practitioner sufficient to complete such obstruction removal procedure, and/or may increase a practitioner's ability to visualize a target area during a procedure. Although primarily discussed in the context of kidney stone removal procedures, any of the systems, devices, and features thereof discussed herein may be used or adapted for use in other parts of the body to remove other materials or obstructions. The medical devices and systems disclosed herein in various examples may include one or more of the features or components described in connection with any of the other examples.

The present disclosure includes a medical system comprising a first medical device. The first medical device may comprise a handle assembly including an actuator; a shaft extending longitudinally from the handle assembly to a distal end; and a wire assembly extending through the shaft. The wire assembly may comprise a proximal portion coupled to the handle assembly and extending longitudinally through the shaft; a first distal extension extending distally from the proximal portion; and a second distal extension extending distally from the proximal portion. A first proximal end of the first distal extension may be integrated with or coupled to a second proximal end of the second distal extension. The shaft may include a proximal section extending around the proximal portion, a first distal section extending around the first extension, a second distal section extending around the second extension, and a basket portion. The basket portion may be configured to transition from a first retracted configuration to a first expanded configuration.

In some aspects, the medical system may include one or more of the following features. In the first expanded configuration, the basket portion may expand radially outward relative to a central longitudinal axis of the shaft, and the basket portion may define a proximally-facing recess in the first expanded configuration. The proximal section, the first distal section, the second distal section, and the basket portion may be fluidly connected to one another. The handle assembly may include a fluid port configured to supply fluid to the shaft to transition the basket portion from the first retracted configuration to the first expanded configuration. The first distal extension and the second distal extension joint together may form a rounded distalmost end. The system may include a second medical device that comprises a sheath configured to extend over a shaft of a third medical device; and a distal balloon portion configured to couple to a distal end of the third medical device, and the distal balloon portion is cylindrical and in fluid communication with the sheath. The distal balloon portion may be configured to transition from a second retracted configuration to a second expanded configuration by expanding distally and radially outward relative to a central longitudinal axis of the shaft.

In some aspects, the medical system may include one or more of the following features. The distal balloon portion may be configured to extend around the first distal section and the second distal section of the first medical device when in the second expanded configuration; and the distal balloon portion may be configured to abut the basket portion of the first medical device when in the second expanded configuration. The second medical device may further comprise an adapter coupled to a proximal end of the sheath, and the adapter may include a lumen and a fluid port. The sheath may include a tube fluidly connecting the distal balloon portion to the adapter. The handle assembly may be fluidly coupled to a fluid source and include a proximalmost aperture configured to receive one or more fingers of a hand of a user. A radially-outermost diameter of the distal balloon portion in the second expanded configuration may be substantially longitudinally aligned with a radially-outermost diameter of the basket portion in the first expanded configuration. The system may include a third medical device, which may be or include a ureteroscope. The shaft of the ureteroscope may extend through the sheath of the second medical device and the shaft of the first medical device extends through a lumen of the ureteroscope. The distal balloon portion may extend circumferentially around a distal end of the ureteroscope. The distal balloon portion and the basket portion may be configured to form a closed interior cavity when the basket portion is in the first expanded configuration and the distal balloon portion is in the second expanded configuration.

In some aspects, the medical system may comprise a first medical device including a shaft, and a second medical device. The second medical device may comprise a sheath configured to extend over the shaft of the first medical device; and a distal balloon portion configured to couple to a distal end of the first medical device, wherein the distal balloon portion is configured to fluidly couple to a fluid source and includes a central lumen configured to extend around the distal end of the first medical device. The distal balloon portion may be configured to transition from a second retracted configuration to a second expanded configuration, the distal balloon portion having a cylindrical shape in the second expanded configuration.

In some aspects, the medical system may include one or more of the following features. A wire assembly may extend through the shaft, e.g., the wire assembly comprising a proximal portion extending longitudinally through the shaft; a first distal extension extending distally from the proximal portion; and a second distal extension extending distally from the proximal portion. A first proximal end of the first distal extension may be integrated with or coupled to a second proximal end of the second distal extension. The shaft may include a proximal section extending around the proximal portion, a first distal section extending around the first extension, a second distal section extending around the second extension, and a basket portion; and the basket portion may be configured to transition from a first retracted configuration to a first expanded configuration. A radially-outermost diameter of the distal balloon portion in the second expanded configuration may be substantially longitudinally aligned with a radially-outermost diameter of the basket portion in the first expanded configuration. The distal balloon portion may be configured to extend around the first distal section and the second distal section of the first medical device when in the second expanded configuration; and the distal balloon portion may be configured to abut the basket portion when in the second expanded configuration.

In some aspects, the present disclosure includes a method that comprises supplying fluid to a shaft of a first medical device to transition a basket portion of the first medical device from a first retracted configuration to a first expanded configuration. The first medical device may comprise a shaft extending longitudinally to a distal end; and a wire assembly extending through the shaft. The wire assembly may comprise a proximal portion extending longitudinally through the shaft; a first distal extension extending distally from the proximal portion; and a second distal extension extending distally from the proximal portion, and a first proximal end of the first distal extension may be integrated with or coupled to a second proximal end of the second distal extension. The method may further include supplying fluid to a second medical device to transition a balloon portion of the second medical device from a second retracted configuration to a second expanded configuration. The balloon portion may be cylindrical and extend around the first distal extension and the second distal extension of the first medical device when in the second expanded configuration. The balloon portion may abut the basket portion of the first medical device when in the second expanded configuration.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, device, or apparatus that comprises a list of elements does not include only those elements, but may include other elements not expressly listed or inherent to such process, method, article, device, or apparatus. The term “exemplary” is used in the sense of “example,” rather than “ideal.” The term “distal” refers to a portion farthest away from a user when introducing a device into a patient. By contrast, the term “proximal” refers to a portion closest to the user when placing the device into the patient. Proximal and distal directions are labeled with arrows marked “P” and “D”, respectively, throughout various figures. Although ureteroscopes are referenced herein, reference to ureteroscopes or urology should not be construed as limiting the possible applications of the disclosed aspects. For example, the disclosed aspects may be used with endoscopes, duodenoscopes, bronchoscopes, colonoscopes, catheters, diagnostic or therapeutic tools or devices, or other types of medical devices. Further, relative terms such as, for example, “about,” “substantially,” “approximately,” etc., are used to indicate a possible variation of ±10% in a stated numeric value or range.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIG. 1 is a perspective view of an exemplary medical system and a portion of a body lumen and kidney, according to aspects of this disclosure.

FIG. 2 is a perspective view of a distal end of a medical device, according to aspects of this disclosure.

FIG. 3 is a side view of a medical device with a magnified distal portion of the medical device, according to aspects of this disclosure.

FIGS. 4A-4D are illustrations of a method of use of the medical device of FIG. 3, according to aspects of this disclosure.

FIG. 5A-5B are illustrations of a distal portion of the medical system of FIG. 1, according to aspects of this disclosure.

FIG. 6 is a perspective view of a portion of a medical device, according to aspects of this disclosure.

FIG. 7 is a perspective view of a portion of a medical device, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts.

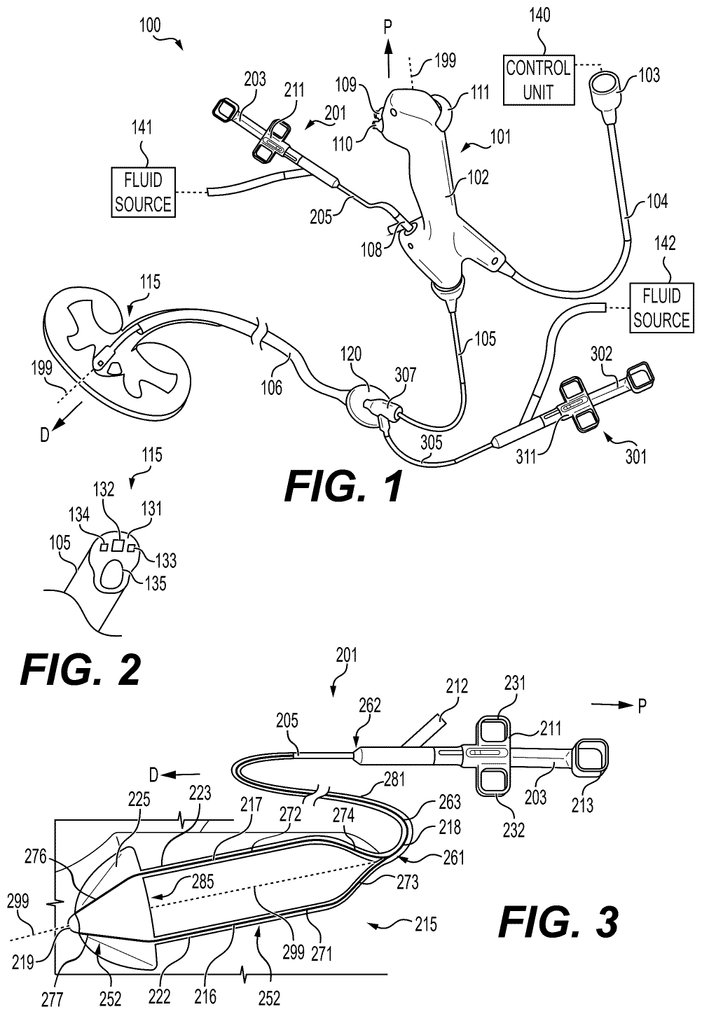

An exemplary medical system 100 is shown in FIG. 1 comprising a ureteroscope 101 (or optionally another scope such as an endoscope). Ureteroscope 101 may include a handle assembly 102 and a shaft 105. Shaft 105 may be sufficiently flexible to allow shaft 105 to navigate through a subject's tortuous anatomical passages. Shaft 105 may terminate at a distal end 115. Shaft 105 may include an articulation section for deflecting distal end 115 in one or more directions (e.g., up, down, left, and/or right directions) and articulation of distal end 115 may be controlled by one or more actuators 109, 110, 111. Medical device 101 may have a central longitudinal axis 199. In at least one example, the articulation section may provide for full retroflexion (e.g., rotation of distal end 115 through an arc of 180 degrees) or only partial retroflexion (e.g., rotation of distal end 115 through an arc of less than 180 degrees). Ureteroscope 101 also may include one or more lumens extending therethrough terminating in corresponding opening(s) 135 (shown in FIG. 2). For example, the one or more lumens may extend through shaft 105 optionally in communication with corresponding lumen(s) of handle assembly 102. For example, a port 108 on handle assembly 102 may include an opening in fluid communication with a lumen of shaft 105, and an opening 135 of the lumen at distal end 115 may be on a distal front face 131 of shaft 105. System 100 also includes a first medical device 201 and a second medical device 301, shown in FIG. 1 coupled to ureteroscope 101, and will be discussed in further detail hereinbelow.

FIG. 2 illustrates a perspective view of a distal end 115 of ureteroscope 101. Distal end 115 may include a distal front face 131, an imaging assembly 132, one or more illuminators 133, 134, and an opening 135 of a lumen extending through shaft 105 of ureteroscope 101, such as an opening of a working channel. Imaging assembly 132 may include an image sensor, a lens assembly, a fiber optic, one or more circuit boards, and/or any other suitable imaging device or supporting electronic or optics. Imaging assembly 132 may include a charge-coupled device (CCD) image sensor, a complementary metal-oxide image semiconductor (CMOS), or the like coupled to a cable or wire running through the shaft 105 of ureteroscope 101. Illuminators 133, 134 may be light emitting diodes (LEDs), fiber optic-based lights, or any other suitable form of light source. In some examples, one or more windows may be in front of the one or more illuminators 133, 134.

In some examples, suction may be applied to lumen of shaft 105 and opening 135 via a vacuum source, and/or a fluid may be supplied to such lumen and opening 135 to provide and/or aspirate fluid through opening 135 via a fluid source, such as a fluid pump with saline or water.

As mentioned above, while aspects of the present disclosure refer to ureteroscope 101, any suitable medical device for insertion into a patient's body, such as, e.g., an endoscope, a gastroscope, a nephroscope, a colonoscope, a hysteroscope, a bronchoscope, a cystoscope, a duodenoscope or other medical device with a catheter may be used herein.

One or more auxiliary devices may be operatively coupled to ureteroscope 101. Exemplary auxiliary devices may include a control unit 140. In some examples, control unit 140 may include an imaging system, a power supply, a display, a fluid supply, and/or a vacuum source, each of which is briefly described below. Control unit 140 may include, for example, any electronic device capable of receiving, storing, processing, generating, and/or transmitting data according to instructions given by one or more programs. Control unit 140 may be operatively coupled to, or part of, one or more of ureteroscope 101 and the other auxiliary devices, to control one or more aspects of their operation. In some examples, ureteroscope 101 may include connector 103 for connecting to control unit 140. A power supply may include any suitable power source, and associated connectors (e.g., electrically conductive wires), for supplying electronic components in the auxiliary devices and ureteroscope 101 with electrical power. A fluid supply, such as fluid sources 141, 142, may include a reservoir, a medical irrigation bag, a pump, and any suitable connectors (e.g., tubing for fluidly coupling fluid supply and ureteroscope 101 or other devices). The pump may supply a flow of pressurized fluid to one or more of the lumens in ureteroscope 101 or other devices of the system 100, and the pressurized fluid flow may be emitted from distal end 115 and/or used to inflate expandable components present at distal end 115. A vacuum source may provide suction or vacuum pressure to one or more lumens of the ureteroscope 101 or other devices of the system 100, and thereby provide a suction force or negative pressure to draw material toward and/or into ureteroscope 101, and/or to deflate expandable components.

Imaging system may include imaging electronics to, for example, process signals received from an image sensor in ureteroscope 101 (e.g. imaging assembly 132), send signals for controlling the imaging assembly 132, adjust illumination levels of areas being viewed by the image assembly 132 (e.g. illuminators 133, 134), and/or facilitate the display of image sensor data on an electronic display.

A sheath 106 may be used to facilitate insertion of shaft 105 into a body of a patient. Sheath 106 may be used to facilitate moving shaft 105 up and down through a bodily lumen (e.g., a ureter) multiple times without reintroduction of guidewires to do so, and may protect the patient's body from potential damage caused by the movement of shaft 105 through the bodily lumen. Sheath 106 may also prevent damage to the ureteroscope 101 during a procedure. In some examples, sheath 106 may have an outer diameter between 10F and 16F. Sheath 106 may include a proximal end frame 120 configured to couple to an exterior portion of the patient, such as at an insertion site on skin of the patient.

A first medical device 201 may be inserted into a working channel or lumen of ureteroscope 101, for example through port 108, and first medical device 201 may exit out of the distal end 115, such as out opening 135. Referring to FIG. 3, which illustrates first medical device 201 removed from ureteroscope 101, first medical device 201 may include a handle 203 including an actuator 211, and a shaft 205 extending longitudinally to a distal end 215. Distal end 215 is shown magnified in FIG. 3 for better illustration of various features. Handle 203 may include an aperture 213 at a proximalmost end of handle 203, and aperture 213 may be configured to receive under input (e.g., one or more fingers of a user) to facilitate in actuation of actuator 211. Actuator 211 may include two apertures 231, 232, and each aperture 231, 232 may be configured to receive one or more fingers to facilitate in actuation of actuator 211. Actuator 211 may be configured to slide longitudinally, in a proximal or distal direction, along handle 203 from a position abutting aperture 213 to a distal portion of handle 203. Handle 203 or a portion of first medical device 201 proximate handle 201 may include a fluid input 212 configured to couple to a fluid source 141, 142 and supply fluid from fluid source 141, 142 to shaft 205.

A wire assembly 218 may extend longitudinally from handle 203 through a central lumen 261 of shaft 205 to distal end 215. Wire assembly 218 may include a proximal section 263, a first distal extension 216, and a second distal extension 217. A proximal end of the proximal section 263 may be fixedly coupled to a distal end 262 of handle 203. The first distal extension 216 may be coupled to the second distal extension 217 at a distalmost end 219 of distal end 215. First distal extension 216 and second distal extension 217 may form a distal portion 252, and may be rounded at distalmost end 219. First distal extension 216 may include a proximal portion 273 extending radially outward from a central longitudinal axis 299 of shaft 205, an intermediate portion 271 extending substantially parallel to central longitudinal axis 299, and a distal portion 277 extending radially-inward towards central longitudinal axis 299. Second distal extension 217 may include a proximal portion 274 extending radially outward from a central longitudinal axis 299 of shaft 205, an intermediate portion 272 extending substantially parallel to central longitudinal axis 299, and a distal portion 276 extending radially-inward towards central longitudinal axis 299.

In some examples, wire assembly 218 may comprise Nitinol or any other appropriate expandable, e.g., shape-memory, material. First distal extension 216 and second distal extension 217 may transition from (i) a collapsed or retracted configuration in which intermediate portions 271, 272 abut each other and both first distal extension 216 and second distal extension 217 are substantially aligned with central longitudinal axis 299 to (ii) an expanded configuration as shown in FIG. 3 in which intermediate portions 271, 272 are spaced from central longitudinal axis 299. First distal extension 216 and second distal extension 217 may be biased towards the expanded configuration, may be transitioned to a collapsed or retracted configuration when inserted into a lumen of shaft 105 of ureteroscope 101 (e.g., due to space constraints of the lumen), and may expand to the expanded configuration when moved distally out of distal opening 135 and unconstrained by ureteroscope 101.

Shaft 205 may include a proximal section 281, a first distal section 222, a second distal section 223, and a basket section 225 at a distal end of shaft 205. Each of the proximal section 281, the first distal section 222, the second distal section 223, and the basket section 225 may be fluidly connected, and wire assembly 218 may be positioned entirely within shaft 205. Each of the proximal section 281, the first distal section 222, and the second distal section 223 may be substantially cylindrical and include a central lumen 261. The proximal section 281 may extend around proximal section 263 of wire assembly 218; the first distal section 222 may extend around the proximal portion 273 and intermediate portion 271 of the first distal extension 216 of wire assembly 218, and the second distal section 223 may extend around the proximal portion 274 and intermediate portion 272 of the second distal extension 217 of wire assembly 218. Basket section 225 may extend around distal portion 277 of the first distal extension 216 and extend around distal portion 276 of the second distal extension 217.

Basket section 225 may be hemispherical, and may include a recess 285 that may be bowl shaped and configured to collect material. Basket section 225 may be configured to transition from a collapsed or retracted configuration to an expanded configuration (shown in FIG. 3) via supply of fluid to lumen 261 of shaft 205. For example, moving actuator 211 distally relative to aperture (proximal ring) 213 may supply fluid to shaft 205 and cause basket section 225 to expand into an expanded configuration, and moving actuator 211 proximally towards proximal ring 213 may suction fluid out of shaft 205 and cause basket section to retract into a retracted configuration.

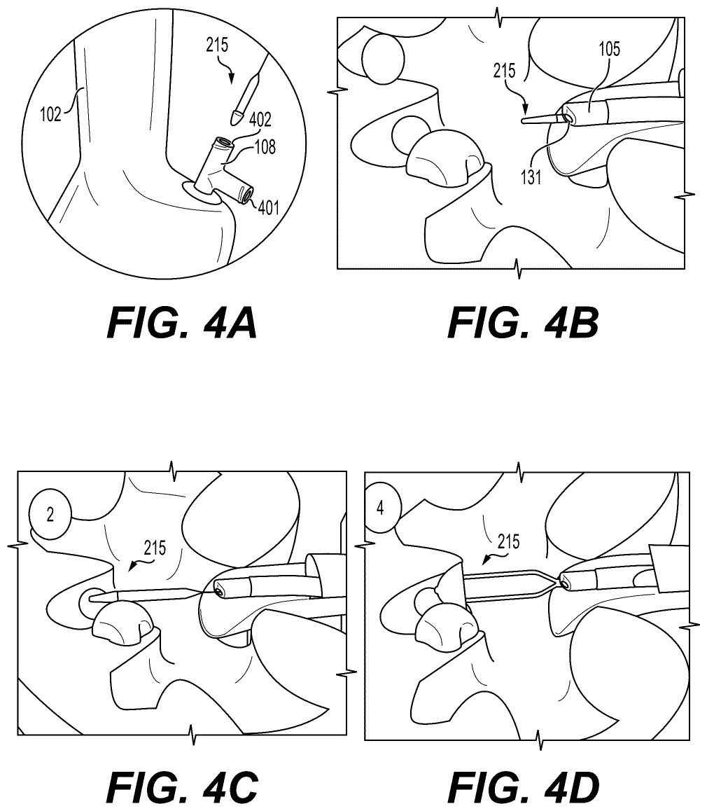

FIGS. 4A-4D illustrate a method of using first medical device 201 and ureteroscope 101. A user may first position distal end 115 of shaft 105 of ureteroscope 101 at a target site of a subject (e.g., patient) for material collection. Once distal end 115 is positioned at the target site, distal end 215 of first medical device 201 is inserted into port 108 of handle assembly 102, as shown in FIG. 4A. Port 108 may have multiple access lumens 401, 402 to allow multiple devices to be inserted into a lumen of ureteroscope 101 at the same time. Distal end 215 is shown in a retracted configuration in FIG. 4A. A user may move distal end 215 distally through shaft 105 to exit shaft out opening 135, as shown in FIG. 4B. Once distal end 215 is fully outside of shaft 105, as shown in FIG. 4C, the user may supply fluid to lumen 261 of shaft 205 to expand basket section 225. When the user supplies fluid to lumen 261, distal end 215 may transition from a retracted configuration (shown in FIG. 4C) to an expanded configuration (shown in FIG. 4D). Once in an expanded configuration, the user may move distal end 215 to collect material within recess 285. In some examples, first medical device 201 may then be removed from the patient with the material after collection of material within recess 285. In other examples, first medical device 201 may be used with second medical device 301 to facilitate collection of material.

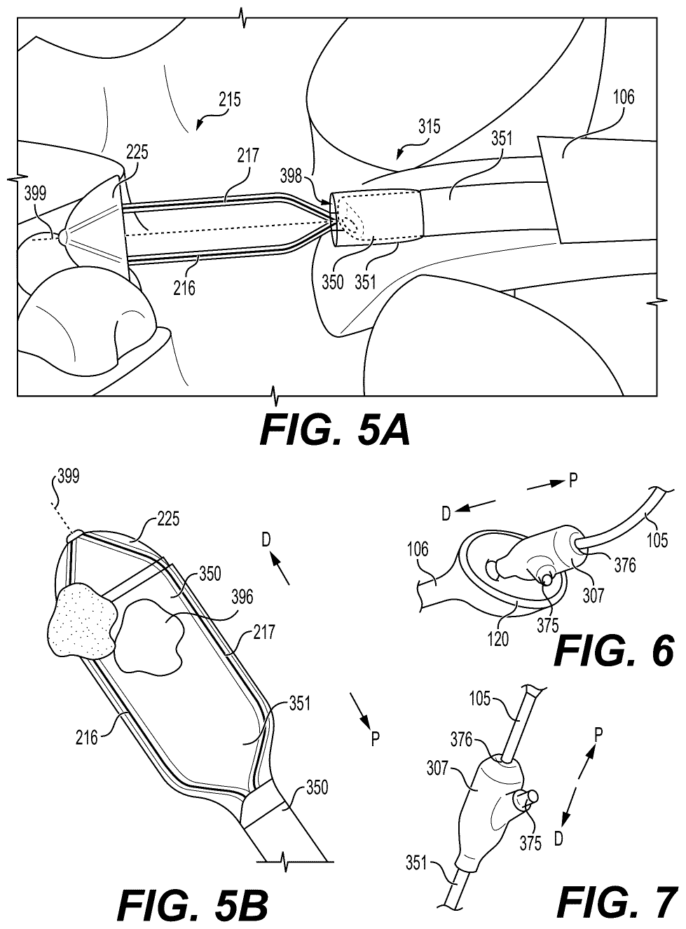

FIG. 5A illustrates distal end 215 of the first medical device 201 in an expanded configuration, and distal portion 315 of the second medical device 301 in a retracted configuration. Second medical device 301 may include a sheath portion 351 including a distal balloon portion 350. Although shown with sheath portion 351 extending over shaft 105 of ureteroscope 101 in FIG. 5A, second medical device 301 may alternatively include a separate tube/shaft extending from distal balloon portion 350 to a fluid source at a proximal portion of the second medical device 301. Sheath portion 351 and distal balloon portion 350 may be fluidly connected. When used with ureteroscope 101, a radially-inward facing surface of distal balloon portion 350 may form a seal with shaft 105 when coupled to shaft 105. Second medical device 301 may include a handle assembly 302 with any of the features of handle assembly 203, and handle assembly 302 may be actuated via actuator 311 to supply fluid to sheath portion 351, for example via connecting portion 305. Distal balloon portion 350 may be cylindrical, may extend over a distalmost portion of shaft 105, and may include a central lumen 398 extending longitudinally through distal balloon portion 350.

When fluid is supplied to sheath portion 351 and distal balloon portion 350, distal balloon portion 350 may expand distally and may expand radially-outwards, relative to longitudinal axis 399. At the same time, distal balloon portion 350 may extend around first extension 216 and second distal extension 217 of wire assembly 218. FIG. 5B illustrates distal balloon portion 350 in a fully expanded configuration with distal balloon portion 350 expanded and extending distally to a proximalmost end of basket section 225. In some examples, distal balloon portion 350 may abut basket section 225 when distal balloon portion 350 is in a fully expanded configuration. As shown in FIG. 5B, distal balloon portion 350 may facilitate capture of material 396 within lumen 398 when distal balloon portion 350 is in an expanded configuration. Distal balloon portion 350, combined with basket section 225, may form an enclosed capsule for material collected with the body of the patient, which a user may then remove from the patient by withdrawing first medical device 201 and second medical device 301 from the patient.

FIG. 6 illustrates a proximal portion of second medical device 301 and shaft 105 of ureteroscope 101 positioned at frame 120 of sheath 106. An adapter 307 of second medical device 301 may facilitate coupling to shaft 105 and to a fluid source 142. For example, adapter 307 includes a central lumen with opening 376 configured to receive shaft 105, and central lumen may be fluidly connected to sheath portion 351 (shown in FIG. 7). A fluid port 375 of adapter 307 may be configured to receive fluid from a fluid source 142, and may be fluidly connected to sheath portion 351. Adapter 307 may be configured to facilitate coupling second medical device 301 to ureteroscope 101.

In operation, medical device system 100 may be used to remove material from a patient's body, and may facilitate a variety of medical procedures such as kidney stone removal procedures. A user may first insert ureteroscope 101 into the adapter 307 and sheath portion 351 of the second medical device 301, and position distal balloon portion 350 over a distal portion of shaft 105. The user may then position distal end 115 of the ureteroscope 101 proximate to a target area within the body of a patient, either with the assistance of a sheath 106 or without sheath 106. The user may then position first medical device 201 within a lumen of shaft 105 and move distal end 215 of the first medical device 201 out of distal opening 135. The user may then expand distal end 215 by supplying fluid to lumen 261 of shaft 205, and thus transitioning distal end 215 to an expanded configuration with basket section 225 expanded to define recess 285, e.g., basket section 225 having a hemispherical shape.

The user may then position material, such as a kidney stone, within or partially within recess 285 of basket section 225, or position the material in longitudinal alignment with basket section 225. The user may then supply fluid to sheath portion 351 via fluid port 375, and expand distal balloon portion 350 to trap the material within distal balloon portion 350 and basket section 225. In some examples, before, during, and/or after capturing the material, a user may manipulate the material, such as by applying laser energy to the material (e.g., with a third medical device positioned with a lumen of shaft 105) to at least partially break apart the material. The combination of distal balloon portion 350 and basket section 225 both in an expanded configuration allows broken parts of material, such as dust and other small fragments, to remain contained within the distal balloon portion 350 and basket section 225 and allows the user to remove all or substantially of the material from the patient's body.

The medical devices, systems, and methods discussed in this disclosure may facilitate better visualization of material within a patient's body, may increase the amount of material that can be removed from a patient during a single procedure, may reduce the procedure time and may reduce the risk of injury to the patient. For example, by using the combination of medical devices described herein, a greater amount of material may be removed from a patient in a single insertion of a ureteroscope into the patient's body, thus reducing the need for repeated entries into the patient's body during a single procedure. The medical device system 100 described herein may allow a user to bring material closer to a distal end 115 of a ureteroscope 101 for application of laser energy, which may improve the effectiveness of fragmenting the material with the laser. In some examples, a user may utilize medical system 100 to capture multiple kidney stones during a single insertion of ureteroscope 101 into the patient.

Any of the medical devices 101, 201, 301 described herein may be made of any suitable material, such as plastic, PEBAX®, or any other suitable material. In any of the above-described examples, medical devices 101, 201, 301 may include one or more components that are metallic, a polymer, machined, formed, stamped, insert molded, or any combination thereof. Any aspect(s) of any one of the above-described examples of medical device 101, 201, 301 may be incorporated into any of the other examples of medical devices 101, 201, 301 described herein.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A medical system, comprising:
a first medical device comprising:
a handle assembly including an actuator;
a shaft extending longitudinally from the handle assembly to a distal end; and
a wire assembly extending through the shaft, wherein the wire assembly comprises:
a proximal portion coupled to the handle assembly and extending longitudinally through the shaft;
a first distal extension extending distally from the proximal portion; and
a second distal extension extending distally from the proximal portion, wherein a first proximal end of the first distal extension is integrated with or coupled to a second proximal end of the second distal extension;
wherein the shaft includes a proximal section extending around the proximal portion, a first distal section extending around the first extension, a second distal section extending around the second extension, and a basket portion; and
wherein the basket portion is configured to transition from a first retracted configuration to a first expanded configuration.

2. The system of claim 1, wherein in the first expanded configuration, the basket portion expands radially outward relative to a central longitudinal axis of the shaft, and wherein the basket portion defines a proximally-facing recess in the first expanded configuration.

3. The system of claim 1, wherein the proximal section, the first distal section, the second distal section, and the basket portion are fluidly connected to one another.

4. The system of claim 1, wherein the handle assembly includes a fluid port configured to supply fluid to the shaft to transition the basket portion from the first retracted configuration to the first expanded configuration.

5. The system of claim 1, wherein the first distal extension and the second distal extension joined together form a rounded distalmost end.

6. The system of claim 5, further comprising:
a second medical device comprising:
a sheath configured to extend over a shaft of a third medical device; and
a distal balloon portion configured to couple to a distal end of the third medical device, wherein the distal balloon portion is cylindrical and in fluid communication with the sheath;
wherein the distal balloon portion is configured to transition from a second retracted configuration to a second expanded configuration by expanding distally and radially outward relative to a central longitudinal axis of the shaft.

7. The system of claim 6, wherein the distal balloon portion is configured to extend around the first distal section and the second distal section of the first medical device when in the second expanded configuration; and wherein the distal balloon portion is configured to abut the basket portion of the first medical device when in the second expanded configuration.

8. The system of claim 6, wherein the second medical device further comprises an adapter coupled to a proximal end of the sheath, wherein the adapter includes a lumen and a fluid port.

9. The system of claim 8, wherein the sheath includes a tube fluidly connecting the distal balloon portion to the adapter.

10. The system of claim 6, wherein the handle assembly is fluidly coupled to a fluid source and includes a proximalmost aperture configured to receive one or more fingers of a hand of a user.

11. The system of claim 6, wherein a radially-outermost diameter of the distal balloon portion in the second expanded configuration is substantially longitudinally aligned with a radially-outermost diameter of the basket portion in the first expanded configuration.

12. The system of claim 6, further comprising the third medical device, wherein the third medical device is a ureteroscope.

13. The system of claim 12, wherein the shaft of the ureteroscope extends through the sheath of the second medical device and the shaft of the first medical device extends through a lumen of the ureteroscope.

14. The system of claim 13, wherein the distal balloon portion extends circumferentially around a distal end of the ureteroscope.

15. The system of claim 6, wherein the distal balloon portion and the basket portion are configured to form a closed interior cavity when the basket portion is in the first expanded configuration and the distal balloon portion is in the second expanded configuration.

16. A medical system comprising:
a first medical device including a shaft; and
a second medical device comprising:
a sheath configured to extend over the shaft of the first medical device; and
a distal balloon portion configured to couple to a distal end of the first medical device, wherein the distal balloon portion is configured to fluidly couple to a fluid source and includes a central lumen configured to extend around the distal end of the first medical device;
wherein the distal balloon portion is configured to transition from a second retracted configuration to a second expanded configuration, the distal balloon portion having a cylindrical shape in the second expanded configuration.

17. The system of claim 16, wherein the first medical device further comprises:
a wire assembly extending through the shaft, wherein the wire assembly comprises:
a proximal portion extending longitudinally through the shaft;
a first distal extension extending distally from the proximal portion; and
a second distal extension extending distally from the proximal portion,
wherein a first proximal end of the first distal extension is integrated with or coupled to a second proximal end of the second distal extension;
wherein the shaft includes a proximal section extending around the proximal portion, a first distal section extending around the first extension, a second distal section extending around the second extension, and a basket portion; and
wherein the basket portion is configured to transition from a first retracted configuration to a first expanded configuration.

18. The system of claim 17, wherein a radially-outermost diameter of the distal balloon portion in the second expanded configuration is substantially longitudinally aligned with a radially-outermost diameter of the basket portion in the first expanded configuration.

19. The system of claim 18, wherein the distal balloon portion is configured to extend around the first distal section and the second distal section of the first medical device when in the second expanded configuration; and wherein the distal balloon portion is configured to abut the basket portion when in the second expanded configuration.

20. A method comprising:
supplying fluid to a shaft of a first medical device to transition a basket portion of the first medical device from a first retracted configuration to a first expanded configuration, wherein the first medical device comprises:
a shaft extending longitudinally to a distal end; and
a wire assembly extending through the shaft, wherein the wire assembly comprises:
a proximal portion extending longitudinally through the shaft;
a first distal extension extending distally from the proximal portion; and
a second distal extension extending distally from the proximal portion, wherein a first proximal end of the first distal extension is integrated with or coupled to a second proximal end of the second distal extension; and
supplying fluid to a second medical device to transition a balloon portion of the second medical device from a second retracted configuration to a second expanded configuration, wherein the balloon portion is cylindrical and extends around the first distal extension and the second distal extension of the first medical device when in the second expanded configuration, and wherein the balloon portion abuts the basket portion of the first medical device when in the second expanded configuration.
